(19) United States
(12) Patent Application Publication
(10) Pub. No.: US 2025/0378921 A1
(43) Pub. Date: Dec. 11, 2025

(54) METHODS AND SYSTEMS FOR A CLINICAL DATA INTERCHANGE FRAMEWORK

(71) Applicant: KONINKLIJKE PHILIPS N.V., EINDHOVEN (NL)

(72) Inventor: YEE Him CHEUNG, Cambridge, MA (US)

(21) Appl. No.: 18/857,876

(22) PCT Filed: Apr. 11, 2023
(86) PCT No.: PCT/EP2023/059369

§ 371 Date: Oct. 18, 2024

Related U.S. Application Data
(60) Provisional application No. 63/332,970, filed on Apr. 20, 2022.

(51) Int. Cl. G16H 10/20

(52) U.S. Cl. CPC G16H 10/20

(57) ABSTRACT

A method (100) for accessing clinical data using a clinical data access system in communication with a plurality of remote clinical data databases comprising two or more different access protocols, comprising: (i) receiving (120) an access request for clinical data in one or more of the remote clinical data databases about one or more subjects; (ii) obtaining (130) linkage information for the one or more subjects; (iii) obtaining (140) one or more data modeling templates that specify protocol-specific data fields to be retrieved from the one or more remote clinical data databases; (iv) instantiating (150) a protocol-specific network socket for an identified one of the plurality of remote clinical data data-bases; (v) retrieving (160) clinical data about the one or more subjects from the identified one of the plurality of remote clinical data databases; and (v) providing (190) the retrieved clinical data about the one or more subjects.

### FIELD OF THE DISCLOSURE

The present disclosure is directed generally to methods and systems for accessing and exchanging clinical data using a clinical data access system.

### BACKGROUND

Electronic clinical data in the healthcare setting has become the norm. As a result, most clinical information is digitized in electronic health record systems. However, there is not a single, standardized format for health records, and there exist many different types and formats of electronic health record systems. For example, many electronic health record systems utilize the Fast Healthcare Interoperability Resource (FHIR) format, created by the Health Level Seven International (HL7) health-care standards organization, to store and communicate medical data. FHIR is a standard that describes data formats and elements (i.e., resources) as well as an API for exchanging electronic health records. However, there are many other common formats for the organization, storage, and communication of clinical data, such as the Consolidated Clinical Document Architecture (C-CDA), among others.

There are many existing healthcare information technology (IT) standards and vendor-specific solutions that aim to facilitate the exchange of clinical data across heterogeneous systems. However, these standards and proprietary solutions are generally not interoperable with each other. For healthcare applications to be able to interact with heterogenous systems supporting different protocols, such as HL7 and FHIR, the software modules for handling data exchanges must be re-implemented for each protocol, resulting in unnecessarily complex software solutions with higher development and maintenance costs.

### SUMMARY OF THE DISCLOSURE

Accordingly, there is a continued need in the art for methods and systems that enable efficient and affordable exchange of clinical data across heterogeneous electronic health record systems.

The present disclosure is directed to inventive methods and systems for accessing clinical data using a clinical data access system. The clinical data access system is in communication with a plurality of remote clinical data databases, the plurality of remote clinical data databases comprising two or more different access protocols and therefore necessitating the exchange of clinical data across heterogeneous electronic health record systems. The clinical data access system further comprises linkage information, comprising (i) information, such as a location and access protocol, regarding each of the one or more remote clinical data databases associated with each of the one or more subjects, and (ii) identifiers for the one or more subjects used for lookup in the one or more remote clinical data databases. The system also comprises a data modeling template registry comprising a plurality of data modeling templates of the clinical data access system utilized to access each of the plurality of remote clinical data databases, wherein a data modeling template comprises at least an identification of the access protocol for the respective remote clinical data database, and a format of the clinical data stored in the respective remote clinical data database. The clinical data access system receives, via a user interface of the clinical data access system, an access request for clinical data about one or more subjects, where the requested clinical data is stored in one or more of the remote clinical data databases. The clinical data access system obtains the linkage information, which also identifies one or more of the plurality of remote clinical data databases comprising the requested clinical data. The system obtains, via a data modeling template registry, one or more data modeling templates that specify protocol-specific data fields to be retrieved from the one or more remote clinical data databases. Once a remote clinical data database is identified, the clinical data access system utilizes the identified linkage information to instantiate a protocol-specific network socket for the identified remote clinical data database. The clinical data access system can now retrieve, via the instantiated protocol-specific network socket, clinical data about the one or more subjects from the remote clinical data database. The system applies any data conversion necessary for the retrieved clinical data. The retrieved clinical data about the one or more subjects can then be provided in response to the user request, via any mechanism for sharing or providing clinical data.

Generally, in one aspect, a method for accessing clinical data using a clinical data access system is provided. The clinical data access system is in communication with a plurality of remote clinical data databases, the plurality of remote clinical data databases comprising two or more different access protocols. The method includes: (i) receiving, via a user interface of the clinical data access system or a client application, an access request for clinical data about one or more subjects, wherein the clinical data is stored in one or more of the remote clinical data databases; (ii) obtaining, via a linkage information registry or via a client application, linkage information for the one or more subjects, wherein the linkage information comprises: (1) information, such as a location and access protocol, regarding each of the one or more remote clinical data databases associated with each of the one or more subjects, and (2) identifiers for the one or more subjects used for lookup in the one or more remote clinical data databases; (iii) obtaining, via a data modeling template registry using a template or template group identifier from a user interface or a remote client application, or directly via a remote client application, one or more data modeling templates that specify protocol-specific data fields to be retrieved from the one or more remote clinical data databases; (iv) instantiating, using the linkage information specifying the one or more remote clinical data databases associated with the one or more subjects, a protocol-specific network socket for an identified one of the plurality of remote clinical data databases; (v) retrieving, via the instantiated protocol-specific network socket, clinical data about the one or more subjects from the identified one of the plurality of remote clinical data databases; and (vi) providing the retrieved clinical data about the one or more subjects.

According to an embodiment, the method further includes, via a data handler and an orchestrator of the clinical data access system using information from the one or more data modeling templates, the steps of: applying data pre-processing, such as data conversion and semantics translation, on the retrieved clinical data; and packaging the retrieved clinical data into a desired output data structure.

According to an embodiment, the method further includes instantiating, using the linkage information for an identified second one of the plurality of remote clinical data databases, a second protocol-specific network socket for the identified second one of the plurality of remote clinical data databases; retrieving, via the instantiated second protocol-specific network socket, clinical data about the one or more subjects from the identified second one of the plurality of remote clinical data databases; and merging and packaging the clinical data retrieved from the identified one of the plurality of remote clinical data databases and the identified second one of the plurality of remote clinical data databases.

According to an embodiment, a user browses and selects, via the user interface or a client application, linkage information for access to the one or more of the plurality of remote clinical data databases comprising the clinical data of the one or more subjects.

According to an embodiment, the method further includes the step of defining or modifying, via the user interface or a defining or modifying tool, linkage information to add linkage information for a new clinical data database or update linkage information for an existing clinical data database, the linkage information comprising at least: (i) information, such as a location and access protocol, regarding each of the one or more remote clinical data databases associated with each of the one or more subjects, and (ii) identifiers for the one or more subjects used for lookup in the one or more remote clinical data databases.

According to an embodiment, the method further includes defining or modifying, via the defining or modifying tool or a user interface, a data modeling template for the clinical data access system. According to an embodiment, defining a data modeling template for the clinical data access system comprises: identifying one or more protocol-specific data fields to be retrieved or updated; specifying data pre-processing to be applied to retrieved data; and specifying how retrieved data fields will be organized and packaged in the output data structure and the data formatting language utilized.

According to an embodiment, the instantiated protocol-specific network socket generates a request to query or update the clinical data in the identified one of the plurality of remote clinical data databases, and wherein the instantiated protocol-specific network socket parses a response received to the query using a messaging format of the designated access protocol.

According to an embodiment, the linkage information further comprises access and/or authentication credentials or encryption/decryption keys for one or more of the plurality of remote clinical data databases.

According to an embodiment, the system further comprises a gateway service that handles dynamic data update requests by listening to and receiving event-triggered push messages from the remote clinical data databases, using the data sockets and data handlers to process the received data, storing the updates locally and sending them to the client application periodically or upon request.

In accordance with another embodiment is a system for accessing clinical data. The clinical data access system is in communication with a plurality of remote clinical data databases, the plurality of remote clinical data databases comprising two or more different access protocols, and further comprising stored clinical data about one or more subjects, the system comprising: a user interface; and a processor configured to: (i) receive, via the user interface system or a client application, an access request for clinical data about one or more subjects, wherein the clinical data is stored in one or more of the plurality of remote clinical data databases; (ii) obtain, via a linkage information registry or via the client application, linkage information for the one or more subjects, wherein the linkage information comprises: (1) information, such as a location and access protocol, regarding each of the one or more remote clinical data databases associated with each of the one or more subjects, and (2) identifiers for the one or more subjects used for lookup in the one or more remote clinical data databases; (iii) obtain, via a data modeling template registry using a template or template group identifier from a user interface or a remote client application, or directly via a remote client application, one or more data modeling templates that specify protocol-specific data fields to be retrieved from the one or more remote clinical data databases; (iv) instantiate, using the linkage information specifying the one or more remote clinical data databases associated with the one or more subjects, a protocol-specific network socket for an identified one of the plurality of remote clinical data databases; (v) retrieve, via the instantiated protocol-specific network socket, clinical data about the one or more subjects from the identified one of the plurality of remote clinical data databases; and (vi) provide the retrieved clinical data about the one or more subjects.

According to an embodiment, the processor is further configured to: instantiate, using the linkage information for an identified second one of the plurality of remote clinical data databases, a second protocol-specific network socket for the identified second one of the plurality of remote clinical data databases; retrieve, via the instantiated second protocol-specific network socket, clinical data about the one or more subjects from the identified second one of the plurality of remote clinical data databases; merge and package the clinical data retrieved from the identified one of the plurality of remote clinical data databases and the identified second one of the plurality of remote clinical data databases.

According to an embodiment a user browses and selects, via a user interface or client application, linkage information for access to the one or more of the plurality of remote clinical data databases comprising the clinical data for the one or more subjects.

According to an embodiment, the processor utilizes the instantiated protocol-specific network socket to generate a request to query or update the clinical data in the identified one of the plurality of remote clinical data databases, and to parse a response received to the query using a messaging format of the designated access protocol.

According to an embodiment, the linkage information further comprises access and/or authentication credentials or encryption/decryption keys for one or more of the plurality of remote clinical data databases.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. The figures showing features and ways of implementing various embodiments and are not to be construed as being limiting to other possible embodiments falling within the scope of the attached claims. Also, the drawings are not necessarily to scale, emphasis instead generally being placed upon illustrating the principles of the various embodiments.

FIG. 1 is a flowchart of a method for accessing clinical data using a clinical data access system, in accordance with an embodiment.

FIG. 2 is a schematic representation of a clinical data access system, in accordance with an embodiment.

FIG. 3 is a flowchart of a method for creating or modifying linkage information or data modeling templates using a clinical data access system, in accordance with an embodiment.

FIG. 4 is a schematic representation of a clinical data access system, in accordance with an embodiment.

### DETAILED DESCRIPTION OF EMBODIMENTS

The present disclosure describes various embodiments of a clinical data interchange framework with a uniform interface for data access, providing protocol-specific data access and processing instructions as specified in external templates. More generally, Applicant has recognized and appreciated that it would be beneficial to provide a method and system to enable efficient and affordable exchange of clinical data across heterogeneous electronic health record systems. A clinical data access system comprises or is in communication with a plurality of remote clinical data databases, the plurality of remote clinical data databases comprising two or more different access protocols and therefore necessitating the exchange of clinical data across heterogeneous electronic health record systems. The clinical data access system further comprises linkage information, comprising (i) information, such as a location and access protocol, regarding each of the one or more remote clinical data databases associated with each of the one or more subjects, and (ii) identifiers for the one or more subjects used for lookup in the one or more remote clinical data databases. The system also comprises a data modeling template registry comprising a plurality of data modeling templates of the clinical data access system utilized to access each of the plurality of remote clinical data databases, wherein a data modeling template comprises at least an identification of the access protocol for the respective remote clinical data database, and a format of the clinical data stored in the respective remote clinical data database. The clinical data access system receives, via a user interface of the clinical data access system, an access request for clinical data about one or more subjects, where the requested clinical data is stored in one or more of the remote clinical data databases. The clinical data access system obtains the linkage information, which also identifies one or more of the plurality of remote clinical data databases comprising the requested clinical data. The system obtains, via a data modeling template registry, one or more data modeling templates that specify protocol-specific data fields to be retrieved from the one or more remote clinical data databases. Once a remote clinical data database is identified, the clinical data access system utilizes the identified linkage information to instantiate a protocol-specific network socket for the identified remote clinical data database. The clinical data access system can now retrieve, via the instantiated protocol-specific network socket, clinical data about the one or more subjects from the remote clinical data database. The system applies any data conversion necessary for the retrieved clinical data. The retrieved clinical data about the one or more subjects can then be provided in response to the user request, via any mechanism for sharing or providing clinical data.

In certain embodiments, researchers and/or healthcare professionals may utilize the clinical data access system to retrieve and share clinical information about subjects or patients. One continuing challenge for exchanging clinical information about subjects or patients among heterogeneous health record systems is that clinical data is stored in different formats within these systems. The methods and systems described or otherwise envisioned herein provide a clinical data interchange framework with a uniform interface for data access. This is achieved by allowing protocol-specific data access and processing instructions to be specified in external templates. To access a designated set of data for an application via multiple protocols, a template can be defined for each protocol specifying the data to be accessed using the proper resource naming of the protocol, the local data structure for holding the clinical data to be processed by the application, and the mappings and any additional processing, such as data type conversion and semantics translation required between the external resources and the local data structure. To communicate with an external repository supporting a known protocol, a template designed for the protocol is loaded and the system can communicate using that template information. Thus, the methods and systems described or otherwise envisioned herein are highly beneficial in applications for exchanging clinical information about subjects or patients among heterogeneous health record systems.

In just one possible non-limiting embodiment, the clinical data exchange framework can be integrated with the MPEG-G standard for genomic data storage and management. Adapting MPEG-G to accommodate well-established technologies in the healthcare IT ecosystem can reduce implementation costs and support a more diverse array of software systems. The seamless exchange of both clinical and genomic data is a critical component for precision medicine informatics solutions.

According to an embodiment, the systems and methods described or otherwise envisioned herein can, in some non-limiting embodiments, be implemented as an element for a commercial product for medical data such as Philips® HealthSuite and IntelliBridge (available from Koninklijke Philips NV, the Netherlands), or as an element for any other commercial product for efficient clinical data interchange, or any suitable system.

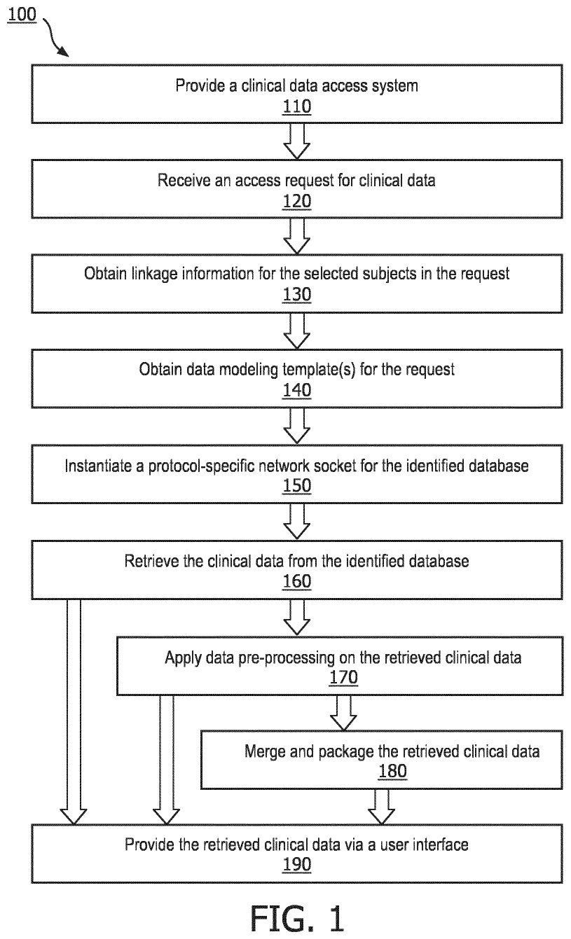

Referring to FIG. 1, in one embodiment, is a flowchart of a method 100 for accessing clinical data. The method can be performed by a clinical data access system. The methods described in connection with the figures are provided as examples only, and shall be understood not to limit the scope of the disclosure. The clinical data access system can be any of the systems described or otherwise envisioned herein. The clinical data access system can be a single system or multiple different systems.

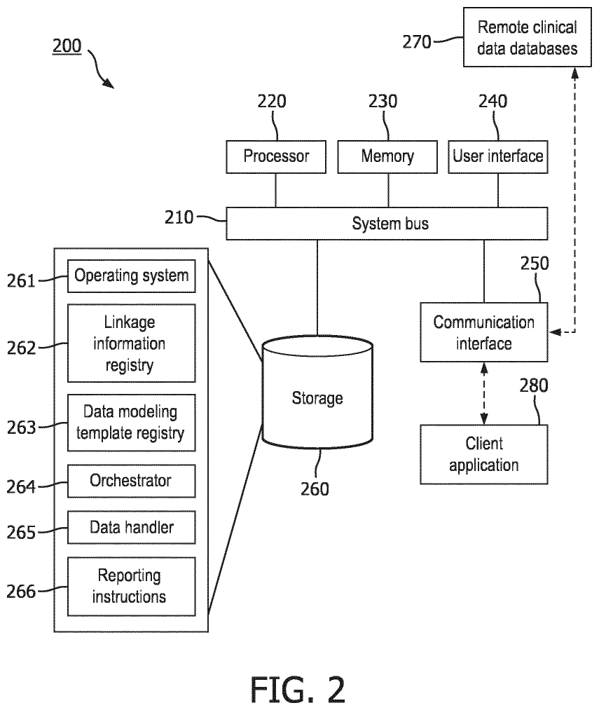

At step 110 of the method, a clinical data access system 200 is provided. Referring to an embodiment of a clinical data access system 200 as depicted in FIG. 2, for example, the system comprises one or more of a processor 220, memory 230, user interface 240, communications interface 250, and storage 260, interconnected via one or more system buses 210. It will be understood that FIG. 2 constitutes, in some respects, an abstraction and that the actual organization of the components of the system 200 may be different and more complex than illustrated. Additionally, clinical data access system 200 can be any of the systems described or otherwise envisioned herein. Other elements and components of system 200 are disclosed and/or envisioned elsewhere herein.

According to an embodiment, clinical data access system 200 comprises or is in communication with a plurality of remote clinical data databases 270, the plurality of remote clinical data databases comprising two or more different access protocols. For example, the remote clinical data databases 270 may be components of a data sharing system or collaborative network or cohort of clinical data or research. Requesting and retrieving clinical data from a remote clinical data database requires a database-specific access protocol. However, given that the remote clinical data databases are components of heterogeneous health record systems, there are different database-specific access protocols within the plurality of remote clinical data databases 270. Accordingly, in order to share or retrieve data from a remote clinical data database, the clinical data access system must identify and utilize the access protocol specific to the remote clinical data database.

According to an embodiment, an access protocol—also called a data exchange protocol—is an interface specification that identifies the content of exchanged data as well as how the data exchange is implemented and managed. As just one example, Fast Healthcare Interoperability Resources (FHIR) is a data exchange protocol that describes clinical data formats and elements (“resources”) and an application programming interface (API) such as a HTTP-based RESTful protocol (i.e., an API that utilizes representation state transfer (REST)) for exchanging clinical data such as electronic health records. With FHIR, clinical data such as medical health records are stored or represented in JSON, XML, or RDF format. That clinical data—formatted in JSON, XML, or RDF format—can be shared or communicated using an API such as a RESTful protocol in which HTTP methods are used to access data or resources in a web application. Other examples of access protocols including HL7, Phenopackets, C-CDA, and others.

One benefit of the methods and systems described or otherwise envisioned herein is that the clinical data access system 200 can interact with any remote clinical data database once the system identifies or is otherwise provided with the access protocol utilized by that remote clinical data database. Thus, in order to communicate with a remote clinical data database, the clinical data access system 200 can first identify the access protocol utilized by the database. To facilitate this identification, the clinical data access system 200 comprises a linkage information registry 262. Although the linkage information registry is shown as a component of storage 260 of the system in FIG. 2, it should be recognized that the linkage information registry could be a remote registry that is in wired and/or wireless communication with system 200.

According to an embodiment, the linkage information registry comprises information utilized by the clinical data access system to retrieve clinical data from one or more remote clinical data databases. To enable this functionality, the linkage information registry comprises an identification of clinical data stored in some or all of the plurality of remote clinical data databases. In other words, the linkage information registry comprises information about, or linkages with, clinical data stored in a remote clinical data database. This identification of clinical data can be information stored in a database table or any other linkage format or mechanism.

As just one example, the linkage information registry can comprise or be in communication with a database of local data entities such as samples in a variant call file or a gene expression file, and can further comprise or be in communication with linkage information that identifies in which of the plurality of remote clinical data databases 270 additional clinical data about the local data entities can be found. The linkage information can also be stored as metadata in a local file, e.g., in the MPEG-G format (the Moving Picture Experts Group—Genomics format for the compression, storage, transmission, and processing of genomic data), or any other format. Thus, according to an embodiment, the linkage information registry comprises an identification of the local data entities (such as sample IDs) for which information can be retrieved, as well as the corresponding matching IDs in the remote clinical data database(s), either as plaintexts or ciphertexts for added security.

Notably, database identification information may be contained or found elsewhere. According to one non-limiting example, a researcher or healthcare professional may already be aware of a target remote clinical data database within which desired information may be found, and thus may identify that database.

According to an embodiment, the linkage information registry further comprises information necessary to facilitate communication between the clinical data access system 200 and the plurality of remote clinical data databases 270. For each remote clinical data database, the linkage information registry can comprise a table or other mechanism for identifying: (i) the data exchange protocol utilized by the database; (ii) the format of exchanged data; and (iii) any access credentials and/or keys for encryption and/or decryption of the exchanged data. As just one non-limiting example, for access to remote clinical data database X, the linkage information registry can identify the data exchange protocol utilized by database X (FHIR), the format of exchanged data from database X (XML), and access credentials (login and password information) for database X. Thus, according to an embodiment, the linkage information comprises access/authentication credentials and encryption/decryption keys for each of one or more of the plurality of remote clinical data databases.

According to an embodiment, clinical data access system 200 comprises or is in communication with a client application 280. The client application may be any application that may utilize the information within and/or managed by system 200. The client application, which may be a local or remote client application, can optionally comprise a user interface, such as user interface 240 or a remote client application user interface. A user may, for example, access clinical data access system 200 via the user interface in order to enter an access request for clinical data about one or more subjects. Client application 280 may also comprise linkage information for the one or more subjects, wherein this linkage information may comprise: (i) information regarding the remote clinical data databases, such as a location and access protocol, associated with each of the one or more subjects, and (ii) the identifiers of the one or more subjects used for lookup in the remote clinical data databases. Client application 280 may also comprise one or multiple data modeling templates that specify the protocol-specific data fields to be retrieved from the remote clinical data databases.

### Linkage Information Registry Example

The following is provided as a non-limiting example of data fields specified for the linkage information. In this non-limiting example, the XML format is utilized. Note that the scopes of the core data components are not limited to the data elements described in this example, and the naming, organization, and format of the data elements need not be strictly followed. The proposed data elements can be expanded or omitted depending on the needs of specific use cases.

As described or otherwise envisioned herein, clinical data linkage information specifies the list of available external data sources, their properties and applicable data modeling templates, and for which samples they contain clinical data. According to an embodiment, the clinical data linkage information consists of two main complex data elements: DataSources and Samples.

According to an embodiment, DataSources is a collection of DataSource elements, each containing the following elements: (i) ID, a unique identifier of the data source; (ii) Name, a name of the data source; (iii) URL, a URL or other identifier of the location of the data source; (iv) Protocol, a data exchange protocol used by the data source such as HL7 and FHIR; (v) AvailableTemplates, a collection of one or multiple Template elements, each specifying the ID of an existing template in the registry that can be used with this data source.

According to an embodiment, Samples is a collection of Sample elements, each containing the following elements: (i) ID, a sample identifier; (ii) AvailableDataSources, a collection of DataSource elements, each specifying the ID of a data source containing clinical data of this sample; (iii) Metadata, a complex structure specifying the external data field values associated with this sample that can be used for query. According to an embodiment, it has an attribute IsEncrypted, if set to true, indicates that all the associated field values are in ciphertext. Otherwise, they are in plaintext. It is a collection of one or multiple Field elements, each with an Id attribute specifying the external data field for query and the unique field value of this sample. And (iv) IsEncrypted, an identifier of encryption. If IsEncrypted is true, then the Metadata value should be encrypted. If IsEncrypted is false, then the Metadata values are not encrypted.

Referring to TABLE 1, in one embodiment, is a non-limiting example of clinical data linkage information.

TABLE 1
Example Clinical Data Linkage Information Specified in XML Format

```
<?xml version=″1.0″ encoding=″UTF-8″?>
<ClinicalDataLinkageInfo>
  <DataSources>
    <DataSource>
      <Id>DS0001</Id>
      <Name>Data Source 1</Name>
      <URL>http://datasource1.com/</URL>
      <AvailableTemplates>
        <Template>T0001</Template>
      </AvailableTemplates>
    </DataSource>
    <DataSource>
      <Id>DS0002</Id>
      <Name>Data Source 2</Name>
      <URL>https://datasource2.com/</URL>
      <AvailableTemplates>
        <Template>T0002</Template>
      </AvailableTemplates>
    </DataSource>
  </DataSources>
  <Samples>
    <Sample>
      <Id>S0001</Id>
      <AvailableDataSources>
        <DataSource>DS0001</DataSource>
        <DataSource>DS0002</DataSource>
      </AvailableDataSources>
      <Metadata IsEncrypted=″true″>
        <Field Id=”Patient.MedicalRecordNumber”>M4uWpNDe5F+Q5l1</Field>
        <Field Id=”Observation.EncounterNumber”>7dtZ+5j9ZTfaXkEyBEfulr0A5P8o=
          </Field>
      </Metadata>
    </Sample>
    <Sample>
      <Id>S0002</Id>
      <AvailableDataSources>
        <DataSource>DS0001</DataSource>
      </AvailableDataSources>
      <Metadata IsEncrypted=″false″>
        <Field Id=”PID.MedicalRecordNumber”>MRN200</Field>
        <Field Id=”PV1.EncounterNumber”>EN200</Field>
      </Metadata>
    </Sample>
  </Samples>
</ClinicalDataLinkageInfo>
```

According to an embodiment, the clinical data access system 200 comprises a template registry 263, also known as a data modeling template registry. Although the template registry 263 is shown as a component of storage 260 of the system in FIG. 2, it should be recognized that the template registry could be a remote registry that is in wired and/or wireless communication with system 200.

According to an embodiment, clinical data linkage information in the linkage information registry specifies an applicable data modeling template from the data modeling template registry 263 for a specific remote clinical data database.

According to an embodiment, a data modeling template in the registry comprises at least an identification of the access protocol for the respective remote clinical data database, and a format of the clinical data stored in the respective remote clinical data database. According to an embodiment, a data modeling template is defined for a specific clinical data interchange protocol and is thus applicable to a clinical data database supporting that protocol. Thus, a data modeling template can comprise one or more of: (i) a designated protocol; (ii) the external data resources/fields to be extracted/updated; (iii) any required data conversion or semantics translation processes between the external and local data sources; (iv) the local data structure with mappings to the external data resources/fields; (v) the local data format (such as XML, JSON, or delimited table format among other examples); and/or (vi) a unique template ID and a template group ID, such that templates of different protocols but sharing the same local data structure and serving the same purpose can be assigned the same template group ID.

As just one non-limiting example, for access to remote clinical data database X, the linkage information registry can identify the data exchange protocol utilized by database X (FHIR), the format of exchanged data from database X (XML), and access credentials (login and password information) for database X.

As just one non-limiting example, the linkage information registry can identify the use of data modeling template A from the data modeling template registry for access to database X. Thus, data modeling template A will comprise the data exchange protocol utilized by database X (FHIR). The data modeling template A can also comprise an identification of one or more data resources or fields from remote database X that will be extracted or updated by the communication from the system, as well as an identification of the local data format of the sample IDs in the clinical data access system (XML in this example), and instructions for converting or translating the data from the format in remote database X to the XML format of the clinical data access system.

### Data Modeling Template Example

The following is provided as a non-limiting example of data fields specified for the data modeling template. In this non-limiting example, the XML format is utilized. Note that the scopes of the core data components are not limited to the data elements described in this example, and the naming, organization, and format of the data elements need not be strictly followed. The proposed data elements can be expanded or omitted depending on the needs of specific use cases.

As described or otherwise envisioned herein, a data modeling template specifies its designated data exchange protocol, the data resources/fields to be extracted from external data repositories, instructions for data mappings and processing, and the output data structure and format. According to one non-limiting embodiment, a data modeling template can contain the following elements: (i) ID, a unique identifier of the template; (ii) GroupId, an ID of its template group, where templates belonging to the same group should share the same local data structure and serve the same purpose; (iii) Name, a name of the template; (iv) Protocol, a clinical data exchange protocol such as FHIR, HL7, Phenopackets, etc.; and (v) Method, an HTTP communication method action type such as ‘Get’, ‘Post’, and so on. The HTTP communication methods can be standard communication methods utilized in the art.

According to an embodiment, the data modeling template can additionally contain a Request element. The Request element is a complex type data structure for specifying the external data fields that can be used for query and filtering. According to an embodiment, the Request element can comprise the following child elements: (i) RequestField, the “Id” attribute that specifies the unique identifier of the external data field that can be used for querying with this template; and (ii) FilteredBy, the “Id” attribute that specifies the unique identifier of the external data field that can be used for filtering the results.

According to an embodiment, the data modeling template can additionally contain a Response element. The Response element is a complex type data structure for specifying the query response data structure, the mappings from the external data resources/fields, and the required data type conversions and translations. According to an embodiment, the Response element can comprise one or multiple ResponseField elements with the following attributes: (i) Id, an ID of the data field in the response data structure; (ii) Source, an ID of the external data resource/field whose data is mapped to this response data field; (iii) SourceDataType, original data type of the external data field; (iv) TargetDataType, target data type of the response data field, data type conversion should be applied if it is different from the source data type; (v) SourceCodingSystem, an original coding system used by the external data field, such as LOINC or SNOMED; and (vi) TargetCodingSystem, the target coding system used by the response data field, semantics translation should be applied if it is different from the source coding system.

Referring to TABLE 2, in one embodiment, is a non-limiting example of a data modeling template.

TABLE 2
Example Data Modeling Templates Specified in XML Format

```
<?xml version=“1.0” encoding=“UTF-8”?>
<Templates>
  <Template>
      <!--unique identifier for the template-->
    <Id>T0001</Id>
      <!--unique identifier for the template-->
    <GroupId>G0001</GroupId>
    <!-- template unique name -->
    <Name>Template 1</Name>
    <!-- Data protocol like FHIR or HL7 -->
    <Protocol>FHIR</Protocol>
    <!--HTTP/HTTPS Method either get or post -->
    <Method>GET</Method>
    <Request>
      <!-- patient unique identifier -->
      <RequestField Id=“Patient.PatientId”></RequestField>
      <!-- Healthcare event during which this observation is made -->
      <RequestField Id=“Observation.EncounterNumber”></RequestField>
      <!-- time-period for observation -->
      <FilteredBy Id=“Observation.EffectiveDateTime”> </FilteredBy>
      <!--Codes identifying names of simple observations-->
      <FilteredBy Id=“Observation.Code”> </FilteredBy>
      <!--Specimen used for this observation-->
      <FilteredBy Id=“Observation.Specimen”> </FilteredBy>
    </Request>
    <Response>
      <ResponseField Id=“MRN” Source=“Patient.MedicalRecordNumber”
        SourceDataType=“String” TargetDataType=“String”></ResponseField>
      <ResponseField Id=“Gender” Source=“Patient.Gender” SourceDataType=“String”
        TargetDataType=“String”></ResponseField>
      <ResponseField Id=“ObsVal” Source=“Observation.ValueString”
        SourceDataType=“String” TargetDataType=“Integer”></ResponseField>
      <ResponseField Id=“Diagnosis” Source=“Encounter.Diagnosis”
        SourceDataType=“String” TargetDataType=“String”
        SourceCodingSystem=“LOINC”
        TargetCodingSystem=“SNOMED”></ResponseField>
    </Response>
  </Template>
  <Template>
    <Id>T0002</Id>
    <GroupId>G0001</GroupId>
    <Name>Template 2</Name>
    <Protocol>HL7</Protocol>
    <Method>TCP/IP</Method>
    <Request>
      <RequestField Id=“PID.PatientId”></RequestField>
      <RequestField Id=“PV1.EncounterNumber”></RequestField>
      <FilteredBy Id=“OBX.EffectiveDateTime”> </FilteredBy>
      <FilteredBy Id=“OBX.Code”> </FilteredBy>
      <FilteredBy Id=“OBX.Specimen”> </FilteredBy>
    </Request>
    <Response>
      <ResponseField Id=“MRN” Source=“PID.MedicalRecordNumber”
        SourceDataType=“String” TargetDataType=“String”></ResponseField>
      <ResponseField Id=“Gender” Source=“PID.Gender” SourceDataType=“String”
        TargetDataType=“String”></ResponseField>
      <ResponseField Id=“ObsVal” Source=“PID.Value” SourceDataType=“String”
        TargetDataType=“String”></ResponseField>
      <ResponseField Id=“Diagnosis” Source=“DG1.DiagnosisText”
        SourceDataType=“String” TargetDataType=“String”
        SourceCodingSystem=“LOINC”
        TargetCodingSystem=“SNOMED”></ResponseField>
    </Response>
  </Template>
</Templates
```

Returning to the method 100 in FIG. 1, at step 120 of the method the clinical data access system receives a request for access to clinical data about one or more subjects, or access to information about one or more samples of those one or more subjects. For example, the clinical data can be medical health records or can be any clinical data utilized by researchers or healthcare professionals, including but not limited to laboratory results, demographics, genomic information, or any other type of medical, research, or other analytic or measurement of a sample or subject.

According to an embodiment, the request for access to the clinical data can be received at the clinical data access system via a user interface 240 of the system. Alternatively, the query or command can be received from a remote system that is in wired and/or wireless communication with the clinical data access system. According to an embodiment, the request for access to the clinical data can be received at the clinical data access system via a user interface of the clinical application 280. Some or all of the clinical data access system may be a cloud-based system. Accordingly, a user such as a clinician, researcher, or healthcare professional may access a command or query user interface via a web portal or application.

According to an embodiment, the request for clinical data comprises an identification of a subject or sample about which more information-from a remote database-will be requested and optionally obtained. For example, a clinician may request any additional information about subject Y from one or more remote databases. As another example, a researcher may request gene expression data from database X, to supplement local sequencing data for one or more subject samples.

According to another embodiment, the request or command is a push from one or more of the remote clinical data databases. For example, a remote clinical data database may receive new information such as clinical data or analytical data for a patient or sample, and be programmed or directed to share that new information with the clinical data access system. Accordingly, the remote clinical data database may send a push to the system to begin communication with the client application(s) that requested dynamic updates from that database.

At step 130 of the method, the clinical data access system 200 obtains, via a linkage information registry 262 or directly via the client application 280, the linkage information of the one or more subjects, wherein linkage information comprises: (i) information regarding the remote clinical data databases, such as a location and access protocol, associated with each of the one or more subjects, and (ii) the identifiers for the one or more subjects used for lookup in the remote clinical data databases. According to an embodiment, a user browses and selects, via the user interface 240 of the system or via the client application 280, linkage information in the linkage information registry or in a remote client for access to the one or more of the plurality of remote clinical data databases comprising the clinical data of the desired subjects.

According to an embodiment, the clinical data access system comprises an orchestrator 264 that facilitates this identification functionality. The orchestrator 264 can be, for example, a module of the clinical data access system, which means therefore that the orchestrator can be a packaged functional hardware unit designed for use with other components, and/or part of a program that performs a particular function of related functions.

At step 140 of the method, the system obtains, via the data modeling template registry 263 using a template or template group identifier from the user interface 240, or directly via the client application, one or multiple data modeling templates that specify the protocol-specific data fields to be retrieved from the remote clinical data databases.

According to an embodiment, the orchestrator is in local or remote wired and/or wireless communication with the linkage information registry 262 and the template registry 263. Upon receiving the query or command from a user or other system, or a push for data sharing from a remote database, the orchestrator utilizes the linkage information registry to identify the one or more of the plurality of remote clinical data databases comprising the clinical data, as well as the linkage information and data modeling template for each of the one or more identified remote clinical data databases.

According to an embodiment, the orchestrator parses the linkage information for the remote database from the linkage information registry, including an identification of the data modeling template for that remote database. The orchestrator loads the appropriate data modeling template(s) defined for the protocols of the identified remote database(s).

At step 150 of the method, the orchestrator utilizes the linkage information specifying the remote clinical data databases associated with the one or more subjects to instantiate a protocol-specific network socket, in order to handle communications with the identified remote clinical data database. According to an embodiment, the network socket is a software structure for a networking architecture, as defined by the application programming interface (API) specific to the system and/or remote database. The protocol-specific network socket may be an internet socket, or may be any other suitable socket to enable communication between the clinical data access system and the remote database. According to an embodiment, the instantiated protocol-specific network socket is created for the communication, and is terminated when the communication is complete or when any other terminating programming enacts, including but not limited to failure to connect, timing, or any other trigger.

At step 160 of the method, the system utilizes the instantiated protocol-specific network socket to communicate with the remote clinical data database in order to access, request, retrieve, or otherwise get the target clinical data from the remote database. According to an embodiment, linkage information for the database from the linkage information registry and data modeling information from the data modeling template specific for the database are utilized by the orchestrator to obtain the requested information.

According to an embodiment, the protocol-specific network socket applies the designated protocol specifications (such as FHIR, among many other protocol specifications) to generate messages, such as query, update or event-triggered push requests, being sent to the external data repositories for a selected data modeling template, and to parse response messages from the external data repository.

According to an embodiment, the instantiated protocol-specific network socket generates a request to query and/or update the clinical data in the identified one of the plurality of remote clinical data databases, and the instantiated protocol-specific network socket parses a response received to the query, using the messaging format of the designated access protocol.

At optional step 170 of the method, the clinical data access system applies any necessary or identified data conversion, translation, mapping, semantics translation, and/or packaging/extraction to the clinical data received from the remote database, according to the instructions defined in a data modeling template. Accordingly, the clinical data access system can comprise a data handler 265 that applies the required data conversion and semantics translations on the results received via the data socket as specified in the template, and merge them together with the local data entities for the same subjects or samples at the clinical data access system. The data handler 265 can be, for example, a module of the clinical data access system, which means therefore that the data handler can be a packaged functional hardware unit designed for use with other components, and/or part of a program that performs a particular function of related functions. According to an embodiment, the orchestrator 264 can direct or control the data handler to perform one or more of its functions.

The retrieved clinical data may be provided in any format the data is in when retrieved from the remote clinical data database. However, at optional step 180 of the method, the clinical data access system 200 packages the retrieved clinical data into the desired output data structure, before providing the retrieved clinical data via the user interface. The desired output data structure may be predetermined or preprogrammed, or the desired output data structure may be defined or otherwise determined by the user via the user interface of the system and/or by the client application 280. For example, the retrieved clinical data may be packaged into a report for the user. Many formats for packaged clinical data, including display of that clinical data, are possible.

At step 190 of the method, the clinical data access system provides or otherwise stores or handles the received clinical data. According to one embodiment, the clinical data access system provides the received clinical data to the clinician, researcher, or healthcare professional via a user interface of the system. According to an embodiment, the system may display a report on a display of the system. Alternatively, the report may be communicated by wired and/or wireless communication to another device. For example, the system may communicate the report to a mobile phone, computer, laptop, wearable device, and/or any other device configured to allow display and/or other communication of the report.

According to an embodiment, the clinical data access system writes the received clinical data to a local database or file. For example, the clinical data access system can store the received clinical data in MPEG-G format in a file or database of the clinical data access system or another system. According to an embodiment, the clinical data access system provides the received clinical data to another application of the clinical data access system or an application of another system that will utilize that data. According to yet another embodiment, the clinical data access system performs two or more of these functions.

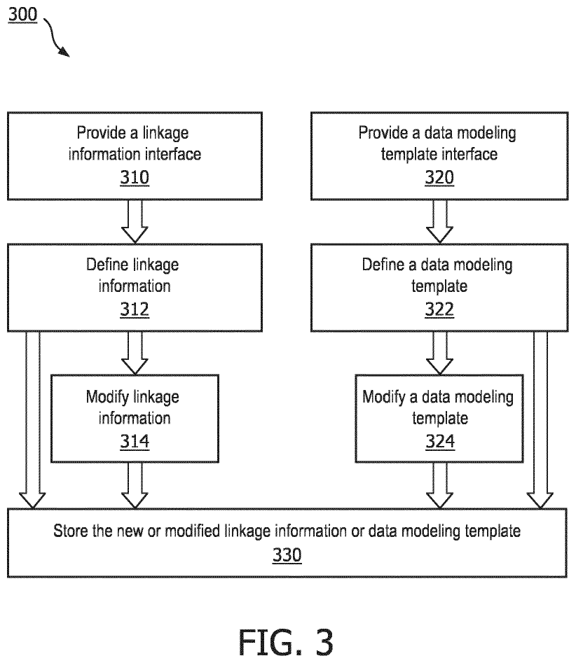

Referring to FIG. 3, in one embodiment, is a method 300 for defining or modifying linkage information in the linkage information registry 262 or the client application 280, and/or defining or modifying a data modeling template in the data modeling template registry 263.

According to an embodiment, at step 310 of the method, a linkage information interface such as user interface 240 or another interface tool, is provided or otherwise accessed by a user. A user such as a clinician, researcher, healthcare professional, or programmer can access the linkage information interface to define linkage information. As described or otherwise envisioned herein, linkage information in the linkage information registry 262 of the clinical data access system 200 comprises information utilized by the clinical data access system to retrieve clinical data from one or more remote clinical data databases. The linkage information may also comprise an identification of the local data entities (such as sample IDs) for which information can be retrieved, as well as the corresponding matching IDs in the remote clinical data database(s), either as plaintexts or ciphertexts for added security. According to an embodiment, the linkage information registry further comprises information necessary to facilitate communication between the clinical data access system 200 and the plurality of remote clinical data databases 270. For each remote clinical data database, the linkage information registry can comprise a table or other mechanism for identifying: (i) the data exchange protocol utilized by the database; (ii) the format of exchanged data; and (iii) any access credentials and/or keys for encryption and/or decryption of the exchanged data.

Accordingly, a user can define any of these elements of the linkage information in the linkage information registry 262 and/or client application 280. At step 312 of the method, for example, a user defines linkage information. This can comprise entering information into a predefined field or otherwise entering or uploading information that populates one or more fields of linkage information in the linkage information registry. As just one example, a user can provide sample IDs for local data entities, as well as the corresponding matching IDs for the samples in a remote clinical data database. As another example, a user can identify a data exchange protocol utilized by a remote clinical data database, as well as a data format utilized by the remote clinical data database. This information can be entered manually or uploaded using standard methods for providing data or datasets.

At optional step 314 of the method, the user can modify any of these elements of the linkage information in the linkage information registry 262. This can comprise modifying existing information about a remote clinical data database, or adding new information about the database. The user can provide the information manually, or the information can be uploaded using standard methods for modifying data or datasets. Modifying linkage information can include, for example, updating the linkage information of an existing clinical data database, and/or adding linkage information for a new clinical data database.

At step 330 of the method, the defined linkage information for the remote clinical data database, and/or modified information for the remote clinical data database, is stored in the linkage information registry 262 of the clinical data access system, where it can be accessed in the future.

According to an embodiment, at step 320 of the method, a data modeling template interface such as via user interface 240, is provided or otherwise accessed by a user. A user such as a clinician, researcher, healthcare professional, or programmer can access the data modeling template interface to define a data modeling template for the data modeling template registry 263. As described or otherwise envisioned herein, a data modeling template in the data modeling template registry 263 of the clinical data access system 200 comprises at least an identification of the access protocol for the respective remote clinical data database, and a format of the clinical data stored in the respective remote clinical data database, among other possible information as described or otherwise envisioned herein. Thus, a data modeling template can comprise one or more of: (i) a designated protocol; (ii) the external data resources/fields to be extracted/updated; (iii) any required data conversion or semantics translation processes between the external and local data sources; (iv) the local data structure with mappings to the external data resources/fields; (v) the local data format (such as XML, JSON, or delimited table format among other examples); and/or (vi) a unique template ID and a template group ID, such that templates of different protocols but sharing the same local data structure and serving the same purpose can be assigned the same template group ID.

Accordingly, a user can define any of these elements of a data modeling template. At step 322 of the method, for example, a user defines one or more data fields of the data modeling template, such as a new template. This can comprise entering information into a predefined field or otherwise entering or uploading information that populates one or more fields of model template information. As just one example, a user can define a designated protocol for a remote clinical data database, as well as the data conversion required to convert data from the clinical database to the format utilized or required by the clinical data access system. This information can be entered manually or uploaded using standard methods for providing data or datasets.

According to an embodiment, therefore defining a new data modeling template in the registry for the clinical data access system comprises: (i) identifying the protocol-specific data fields to be retrieved or updated; (ii) specifying the data pre-processing (e.g. data conversion or semantics translation) that needs to be applied on the retrieved data; and (iii) specifying how the retrieved data fields should be organized and packaged in the output data structure and the data formatting language, such as XML or JSON, to use.

At optional step 324 of the method, the user can modify any of these elements of a data modeling template in the data modeling template registry 263. This can comprise modifying existing information about a remote clinical data database, or adding new information about the database. The user can provide the information manually, or the information can be uploaded using standard methods for modifying data or datasets.

At step 330 of the method, the new data modeling template, and/or the modified data modeling template, is stored in the data modeling template registry 263 of the clinical data access system, where it can be accessed in the future.

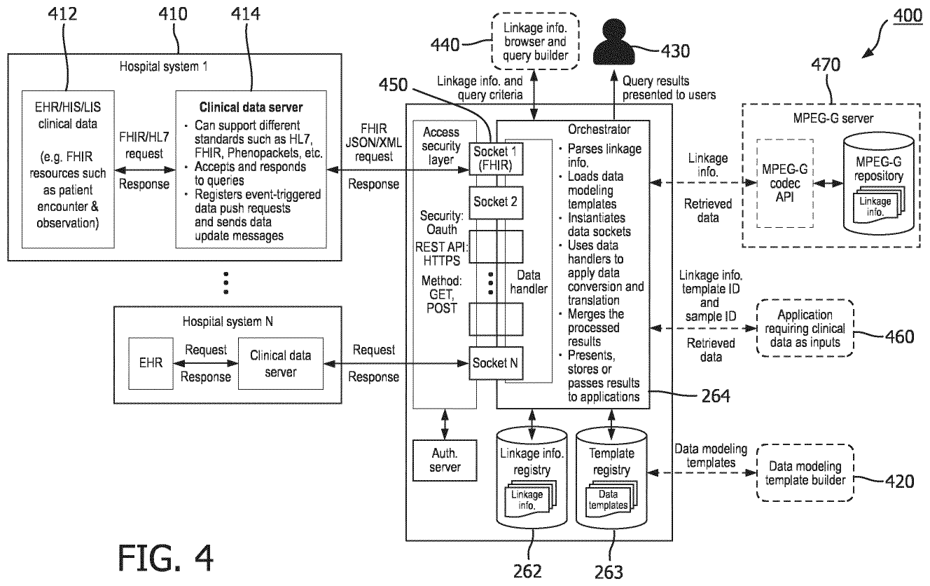

Referring to FIG. 4, in one embodiment, is a schematic representation 400 of a clinical data access system and method, including different functional components and their relationships. In accordance with one non-limiting example, the schematic representation also illustrates how the framework can be integrated with MPEG-G servers, hospital systems, and applications that require clinical data as inputs. Note that linkage information can be stored directly within an MPEG-G dataset as metadata or linkage attributes, rather than in a separate registry, to indicate the availability of additional clinical data in external repositories and the means to access them for specific samples. By making linkage information an intrinsic part of an MPEG-G dataset, it ensures the ready support for clinical data exploration and the linkage information will not be lost during file transport or system migration. Although FIG. 4 provides the use of MPEG-G as an example, it is recognized that many other applications and examples are possible.

In FIG. 4, a hospital system 400 (which can be a single hospital “Hospital System 1” or a plurality of hospitals “Hospital System 1” . . . “Hospital System N”) comprises two components: (i) a backend EHR/HIS/LIS system 412 communicating in some proprietary protocols, and (ii) a clinical data server 414 that interacts with the backend system and connects it to the external environment. According to an embodiment, the clinical data server can be omitted if the EHR/HIS/LIS system can directly communicate with external applications using commonly accepted data exchange protocols.

According to an embodiment, the clinical data server provides the following functionalities: (i) translating and reformatting the query/update/response messages between the internal and external protocols; (ii) performing message validation and discarding any invalid messages; (iii) keeping track of any active dynamic update requests of external clients, and in response posting push-in updates (e.g. a FHIR bundle) to the corresponding data interchange gateways upon receiving relevant event-triggered messages from the backend system; and (iv) authenticating and authorizing data access requests as needed.

Referring again to FIG. 4, the clinical data access system comprises an orchestrator 264 which is in communication with a linkage information registry 262 comprising linkage information, and in communication with a data modeling template registry 263 comprising a plurality of data modeling templates. As described in conjunction with FIG. 3, a user may define or modify a data modeling template. Thus, the system can comprise a component or module such as a data modeling template builder 420 for interfacing with a data modeling template, enabling the building of a new template or modification of an existing template.

Also in FIG. 4, a user 430 can interact with the clinical data access system via a user interface, which may be for example a linkage information browser and/or query builder 440. The linkage information browser and/or query builder 440 allows the user to look for or otherwise identify clinical data that can be retrieved, as well as to identify or select the linkage information necessary to retrieve the clinical data.

The system may further comprise another application 460, or be in communication with a remote application 460, that utilizes clinical data retrieved from the one or more remote databases 410. Similarly, the system may further comprise or be in communication with an MPEG-G server 470, when utilized for that specific application.

Referring to FIG. 4, according to an embodiment, available linkage information can be extracted from a registry or dataset. A user 430 can browse this linkage information in the linkage information registry 262, including the availability of clinical data in one or more remote clinical data databases 410 for specific samples as well as the applicable data modeling templates in the template registry 263. The user can select one or more samples for clinical data retrieval and the data modeling template to apply. According to an embodiment, the orchestrator parses the linkage information associated with the selected samples and instantiates one or multiple protocol-specific network sockets 450, with linkage and template information dispatched, for handling communications with external repositories. The network sockets 450 generate query messages based on the protocols of the target servers and using relevant linkage and template information, and parse query response messages received from external repositories. According to an embodiment, the orchestrator utilizes a data handler to apply any required data conversion and semantics translations on the results from data sockets as specified in the template, and merge them together. The merged results are presented to the user and/or stored in a dataset.

### Application Examples

The following non-limiting examples are provided solely to show several applications or embodiments of the methods described or otherwise envisioned herein, and thus do not limit the scope of the application or the claims.

### Example 1—Exploring External Clinical Data Available for a Dataset

In this example, a user explores a dataset of gene expressions in an MPEG-G file that contains linkage information for specific samples with additional clinical data that could be retrieved from one or more remote clinical data databases. The samples of the dataset are patients under a clinical trial for determining if there exists a group of genes that can predict the treatment response three months after the administration of the first dose of drug based on their expression levels measured before the treatment. Three months have passed after the gene expression data was generated and now the user wants to collect the latest clinical observations, including heart rate and blood pressure. The user identifies that one or more of the samples have clinical data available in one or more remote clinical data databases and there is an applicable Data Modeling Template for retrieving the required clinical data. The user then selects all samples and the data modeling template, specifies that the query results should be stored as attributes associated with the samples of the dataset, and submits a query. Within a few minutes query responses are received from the one or more remote clinical data databases. The system automatically performs data parsing, conversion, translation, merging, and formatting according to the protocol of the database and the information in the selected Data Modeling Template. This results in the generation of the required clinical data of all samples, which are then stored as sample attributes in the dataset using the MPEG-G codec. With the latest and accurate clinical data readily available through the data interchange framework, it removes the burden for finding, downloading and preparing the required clinical data, thus allowing the user to focus on analysis.

### Example 2—Enabling Live Updates to a Local Dataset From External Clinical Data Repositories

In this example, the primary investigator (PI) of a clinical study has an MPEG-G dataset containing linkage information for accessing additional clinical data of the samples in one or more remote clinical data databases. There are a few hundred samples in the dataset and the PI wants to monitor the dynamic trends and statistics of certain health data of all samples on a daily basis over the course of three months without the hassle of manually retrieving and updating the dataset every day. Using the clinical data access system, the PI can submit a dynamic update request with linkage information, patient IDs, and the desired data modeling template. The gateway then registers dynamic update requests with the one or more remote clinical data databases, which will send event-triggered data push-in requests to the gateway when new observation data is added for a patient being monitored. The clinical data access system collects the push-in updates, then on a regular basis processes and merges the new data, and writes them into the dataset using the MPEG-G codec.

For example, according to an embodiment, the system comprises a gateway service that handles dynamic data update requests by listening to and receiving event-triggered push messages from the remote clinical data databases, using the data sockets and data handlers to process the received data, storing the updates locally and sending them to the client application periodically or upon request.

### Example 3—Easy Integration of Live Clinical Data Into Healthcare Applications

In this example, the software architect of a health tech company is looking for an easy solution for incorporating into their clinical decision support system live clinical data of patients from multiple remote clinical data databases using different data exchange standards and vendor-specific solutions. The user decides to use the clinical data access system to handle the extraction and pre-processing of clinical data. A software developer in the team selects a data exchange protocol that needs to be handled. In response, the tool shows a list of available data resources for the selected protocol. The software developer then selects the data fields that are required for a particular software module, and specifies the field names, organization and format (e.g. JSON) of the output data structure, together with mappings to the external data fields and the required data type conversion and translation processes. After clicking the “build” button, a data modeling template is generated. Using the generated template and with proper linkage information and the patient ID, the system can immediately retrieve the clinical data of the patient and output them in a format that can be used directly by the software module. In this way, the problem of integrating live clinical data into a software solution is reduced to simply defining protocol-specific data modeling templates and coupling them to the software modules that use the extracted clinical data in a predefined format.

Referring to FIG. 2, in accordance with an embodiment, is a schematic representation of a clinical data access system 200. System 200 may be any of the systems described or otherwise envisioned herein, and may comprise any of the components described or otherwise envisioned herein. It will be understood that FIG. 2 constitutes, in some respects, an abstraction and that the actual organization of the components of the system 200 may be different and more complex than illustrated.

According to an embodiment, system 200 comprises a processor 220 capable of executing instructions stored in memory 230 or storage 260 or otherwise processing data to, for example, perform one or more steps of the method. Processor 220 may be formed of one or multiple modules. Processor 220 may take any suitable form, including but not limited to a microprocessor, microcontroller, multiple microcontrollers, circuitry, field programmable gate array (FPGA), application-specific integrated circuit (ASIC), a single processor, or plural processors.

Memory 230 can take any suitable form, including a non-volatile memory and/or RAM. The memory 230 may include various memories such as, for example L1, L2, or L3 cache or system memory. As such, the memory 230 may include static random access memory (SRAM), dynamic RAM (DRAM), flash memory, read only memory (ROM), or other similar memory devices. The memory can store, among other things, an operating system. The RAM is used by the processor for the temporary storage of data. According to an embodiment, an operating system may contain code which, when executed by the processor, controls operation of one or more components of system 200. It will be apparent that, in embodiments where the processor implements one or more of the functions described herein in hardware, the software described as corresponding to such functionality in other embodiments may be omitted.

User interface 240 may include one or more devices for enabling communication with a user. The user interface can be any device or system that allows information to be conveyed and/or received, and may include a display, a mouse, and/or a keyboard for receiving user commands. In some embodiments, user interface 240 may include a command line interface or graphical user interface that may be presented to a remote terminal via communication interface 250. The user interface may be located with one or more other components of the system, or may located remote from the system and in communication via a wired and/or wireless communications network.

Communication interface 250 may include one or more devices for enabling communication with other hardware devices. For example, communication interface 250 may include a network interface card (NIC) configured to communicate according to the Ethernet protocol. Additionally, communication interface 250 may implement a TCP/IP stack for communication according to the TCP/IP protocols. Various alternative or additional hardware or configurations for communication interface 250 will be apparent.

Storage 260 may include one or more machine-readable storage media such as read-only memory (ROM), random-access memory (RAM), magnetic disk storage media, optical storage media, flash-memory devices, or similar storage media. In various embodiments, storage 260 may store instructions for execution by processor 220 or data upon which processor 220 may operate. For example, storage 260 may store an operating system 261 for controlling various operations of system 200.

It will be apparent that various information described as stored in storage 260 may be additionally or alternatively stored in memory 230. In this respect, memory 230 may also be considered to constitute a storage device and storage 260 may be considered a memory. Various other arrangements will be apparent. Further, memory 230 and storage 260 may both be considered to be non-transitory machine-readable media. As used herein, the term non-transitory will be understood to exclude transitory signals but to include all forms of storage, including both volatile and non-volatile memories.

While system 200 is shown as including one of each described component, the various components may be duplicated in various embodiments. For example, processor 220 may include multiple microprocessors that are configured to independently execute the methods described herein or are configured to perform steps or subroutines of the methods described herein such that the multiple processors cooperate to achieve the functionality described herein. Further, where one or more components of system 200 is implemented in a cloud computing system, the various hardware components may belong to separate physical systems. For example, processor 220 may include a first processor in a first server and a second processor in a second server. Many other variations and configurations are possible.

The system may comprise or be in communication with a plurality of remote clinical data databases 270, the plurality of remote clinical data databases comprising two or more different access protocols. For example, the remote clinical data databases 270 may be components of a data sharing system or collaborative network or cohort of clinical data or research. Requesting and retrieving clinical data from a remote clinical data database requires a database-specific access protocol. However, given that the remote clinical data databases are components of heterogeneous health record systems, there are different database-specific access protocols within the plurality of remote clinical data databases 270. Accordingly, in order to share or retrieve data from a remote clinical data database, the clinical data access system must identify and utilize the access protocol specific to the remote clinical data database.

The system may comprise or be in communication with a client application 280. The client application may be any application that may utilize the information within and/or managed by system 200. The client application, which may be a local or remote client application, can optionally comprise a user interface, such as user interface 240 or a remote client application user interface.

According to an embodiment, storage 260 of system 200 may store one or more algorithms, modules, and/or instructions to carry out one or more functions or steps of the methods described or otherwise envisioned herein. For example, the system may comprise, among other instructions or data, a linkage information registry 262, a data modeling template registry 263, an orchestrator 264, a data handler 265, and/or reporting instructions 266, among many other possible instructions and/or data.

According to an embodiment, linkage information registry 262 comprises linkage information for a plurality of remote clinical data databases. As described or otherwise envisioned herein, linkage information in the linkage information registry 262 of the clinical data access system 200 comprises information utilized by the clinical data access system to retrieve clinical data from one or more remote clinical data databases. The linkage information may also comprise an identification of the local data entities (such as sample IDs) for which information can be retrieved, as well as the corresponding matching IDs in the remote clinical data database(s), either as plaintexts or ciphertexts for added security. According to an embodiment, the linkage information registry further comprises information necessary to facilitate communication between the clinical data access system 200 and the plurality of remote clinical data databases 270. For each remote clinical data database, the linkage information registry can comprise a table or other mechanism for identifying: (i) the data exchange protocol utilized by the database; (ii) the format of exchanged data; and (iii) any access credentials and/or keys for encryption and/or decryption of the exchanged data.

According to an embodiment, data modeling template registry 263 comprises a plurality of data modeling templates. According to an embodiment, a data modeling template in the registry comprises at least an identification of the access protocol for the respective remote clinical data database, and a format of the clinical data stored in the respective remote clinical data database. According to an embodiment, a data modeling template is defined for a specific clinical data interchange protocol and is thus applicable to a clinical data database supporting that protocol. Thus, a data modeling template can comprise one or more of: (i) a designated protocol; (ii) the external data resources/fields to be extracted/updated; (iii) any required data conversion or semantics translation processes between the external and local data sources; (iv) the local data structure with mappings to the external data resources/fields; (v) the local data format (such as XML, JSON, or delimited table format among other examples); and/or (vi) a unique template ID and a template group ID, such that templates of different protocols but sharing the same local data structure and serving the same purpose can be assigned the same template group ID.

According to an embodiment, orchestrator 264 is a component or module of the clinical data access system that identifies one or more of the plurality of remote clinical data databases 270 comprising requested clinical data, using the linkage information registry 262. The orchestrator also instantiates protocol-specific network sockets for the remote clinical data database, enabling communication of clinical data about the one or more subjects from the remote clinical data database. The orchestrator may also direct the data handler 265 to perform some or all of its functionality.

According to an embodiment, data handler 265 applies any necessary or identified data conversion, translation, mapping, and/or packaging/extraction to the clinical data received from the remote database, according to the instructions defined in a data modeling template, and merges the data together with the local data entities for the same subjects or samples at the clinical data access system. The data handler 265 can be, for example, a module of the clinical data access system, which means therefore that the data handler can be a packaged functional hardware unit designed for use with other components, and/or part of a program that performs a particular function of related functions. According to an embodiment, the orchestrator 264 can direct or control the data handler to perform one or more of its functions.

According to an embodiment, reporting instructions 266 direct the system to provide information retrieved from the one or more remote clinical data databases to a user via a user interface, or to write the data to a file, or to provide the data to another application. According to an embodiment, the system may display a report on a display of the system. The display may comprise information about the identified data or any other information. Alternatively, the report may be communicated by wired and/or wireless communication to another device. For example, the system may communicate the report to a mobile phone, computer, laptop, wearable device, and/or any other device configured to allow display and/or other communication of the report.

According to an embodiment, the clinical data access system is configured to process many thousands or millions of data items, including to identify linking information from the linkage information registry, identify a data modeling template from the data modeling template registration, to instantiate a network socket for communication, to send a request to one or more remote clinical data databases, to receive a response to the query, and to process the data in the received response to provide information to the user and/or to write to a local file and/or to provide the data to another application. This requires processing of millions of datapoints. Thus, retrieving this remote data and utilizing it locally comprises a process with a volume of calculation and analysis that a human brain cannot accomplish in a lifetime, or multiple lifetimes. Further, by providing faster and more efficient method for communicating between heterogenous data systems, the methods and systems described or otherwise envisioned herein represent a technological improvement over previous systems that are not capable of performing the same functionality in the same way.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

1. A method for accessing clinical data using a clinical data access system, wherein the clinical data access system is in communication with a plurality of remote clinical data databases, the plurality of remote clinical data databases comprising two or more different access protocols, the method comprising:
receiving, via a user interface of the clinical data access system or a client application, an access request for clinical data about one or more subjects, wherein the clinical data is stored in one or more of the remote clinical data databases;
obtaining, via a linkage information registry or via a client application, linkage information for the one or more subjects, wherein the linkage information comprises: (i) information, comprising a location and access protocol, regarding each of the one or more remote clinical data databases associated with each of the one or more subjects, and (ii) identifiers for the one or more subjects used for lookup in the one or more remote clinical data databases;
obtaining, via a data modeling template registry using a template or template group identifier from a user interface or a remote client application, or directly via a remote client application, one or more protocol-specific data modeling templates that each specify, for a respective access protocol of the one or more remote clinical data databases, protocol-specific data fields to be retrieved;
instantiating, using the linkage information specifying the one or more remote clinical data databases associated with the one or more subjects, a protocol-specific network socket for an identified one of the plurality of remote clinical data databases;
retrieving, via the instantiated protocol-specific network socket, clinical data contained in the protocol-specific data fields about the one or more subjects from the identified one of the plurality of remote clinical data databases; and
providing the retrieved clinical data about the one or more subjects.

2. The method of claim 1, further comprising, via a data handler and an orchestrator of the clinical data access system using information from the one or more data modeling templates, the steps of:
applying data pre-processing, such as data conversion and semantics translation, on the retrieved clinical data; and
packaging the retrieved clinical data into a desired output data structure.

3. The method of claim 1, further comprising:
instantiating, using the linkage information for an identified second one of the plurality of remote clinical data databases, a second protocol-specific network socket for the identified second one of the plurality of remote clinical data databases;
retrieving, via the instantiated second protocol-specific network socket, clinical data contained in the protocol-specific data fields about the one or more subjects from the identified second one of the plurality of remote clinical data databases; and
merging and packaging the clinical data retrieved from the identified one of the plurality of remote clinical data databases and the identified second one of the plurality of remote clinical data databases.

4. The method of claim 1, wherein a user browses and selects, via the user interface or a client application, linkage information for access to the one or more of the plurality of remote clinical data databases comprising the clinical data of the one or more subjects.

5. The method of claim 1, further comprising the step of defining or modifying, via the user interface or a defining or modifying tool, linkage information to add linkage information for a new clinical data database or update linkage information for an existing clinical data database, the linkage information comprising at least: (i) information, comprising a location and access protocol, regarding each of the one or more remote clinical data databases associated with each of the one or more subjects, and (ii) identifiers for the one or more subjects used for lookup in the one or more remote clinical data databases.

6. The method of claim 1, further comprising the step of defining or modifying, via the defining or modifying tool or a user interface, a data modeling template for the clinical data access system.

7. The method of claim 6, wherein defining a data modeling template for the clinical data access system comprises:
identifying one or more protocol-specific data fields to be retrieved or updated;
specifying data pre-processing to be applied to retrieved data; and
specifying how retrieved data fields will be organized and packaged in the output data structure and the data formatting language utilized.

8. The method of claim 1, wherein the instantiated protocol-specific network socket generates a request to query or update the clinical data in the identified one of the plurality of remote clinical data databases, and wherein the instantiated protocol-specific network socket parses a response received to the query using a messaging format of the designated access protocol.

9. The method of claim 1, wherein the linkage information further comprises access and/or authentication credentials or encryption/decryption keys for one or more of the plurality of remote clinical data databases.

10. The method of claim 1, wherein the system further comprises a gateway service that handles dynamic data update requests by listening to and receiving event-triggered push messages from the remote clinical data databases, using the data sockets and data handlers to process the received data, storing the updates locally and sending them to the client application periodically or upon request.

11. A system for accessing clinical data, wherein the clinical data access system is in communication with a plurality of remote clinical data databases, the plurality of remote clinical data databases comprising two or more different access protocols, and further comprising stored clinical data about one or more subjects, the system comprising:
a user interface; and
a processor configured to: (i) receive, via the user interface system or a client application, an access request for clinical data about one or more subjects, wherein the clinical data is stored in one or more of the plurality of remote clinical data databases; (ii) obtain, via a linkage information registry or via the client application, linkage information for the one or more subjects, wherein the linkage information comprises: (1) information, comprising a location and access protocol, regarding each of the one or more remote clinical data databases associated with each of the one or more subjects, and (2) identifiers for the one or more subjects used for lookup in the one or more remote clinical data databases; (iii) obtain, via a data modeling template registry using a template or template group identifier from a user interface or a remote client application, or directly via a remote client application, one or more protocol-specific data modeling templates that specify, for access protocols of the one or more remote clinical data databases, protocol-specific data fields to be retrieved; (iv) instantiate, using the linkage information specifying the one or more remote clinical data databases associated with the one or more subjects, a protocol-specific network socket for an identified one of the plurality of remote clinical data databases; (v) retrieve, via the instantiated protocol-specific network socket, clinical data contained in the protocol-specific data fields about the one or more subjects from the identified one of the plurality of remote clinical data databases; and (vi) provide the retrieved clinical data about the one or more subjects.

12. The system of claim 11, wherein the processor is further configured to: instantiate, using the linkage information for an identified second one of the plurality of remote clinical data databases, a second protocol-specific network socket for the identified second one of the plurality of remote clinical data databases; retrieve, via the instantiated second protocol-specific network socket, clinical data contained in the protocol-specific data fields about the one or more subjects from the identified second one of the plurality of remote clinical data databases; merge and package the clinical data retrieved from the identified one of the plurality of remote clinical data databases and the identified second one of the plurality of remote clinical data databases.

13. The system of claim 11, wherein a user browses and selects, via the user interface or client application, linkage information for access to the one or more of the plurality of remote clinical data databases comprising the clinical data for the one or more subjects.

14. The system of claim 11, wherein the processor utilizes the instantiated protocol-specific network socket to generate a request to query or update the clinical data in the identified one of the plurality of remote clinical data databases, and to parse a response received to the query using a messaging format of the designated access protocol.

15. The system of claim 11, wherein the linkage information further comprises access and/or authentication credentials or encryption/decryption keys for one or more of the plurality of remote clinical data databases.
